(19) United States
(12) Patent Application Publication
(10) Pub. No.: US 2005/0113878 A1
(43) Pub. Date: May 26, 2005

(54) Method, system and device for treating various disorders of the pelvic floor by electrical stimulation of the pudendal nerves and the sacral nerves at different sites

(75) Inventor: Martin T. Gerber, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/723,903

(22) Filed: Nov. 26, 2003

(51) Int. Cl. A61N 1/18

(52) U.S. Cl. 607/39

(57) ABSTRACT

Described are implantable devices and methods for treating various disorders of the pelvic floor by means of electrical stimulation of the pudendal and sacral nerves, or portions thereof, and optional means for delivering drugs in association therewith. Two or more electrical stimulation regimes are applied on a continuous, alternating, intermittent or other basis to the sacral and pudendal nerves, and optionally one or more drugs are infused, injected or otherwise administered, to appropriate portions of a patient's pelvic floor and pudendal nerve and/or sacral nerve, or portions thereof, in an amount and manner effective to treat a number of disorders, including, but not limited to, urinary and/or fecal voiding dysfunctions such as constipation, incontinence disorders such as urge frequency and urinary retention disorders, sexual dysfunctions such as orgasmic and erectile dysfunction, pelvic pain, prostatitis, prostatalgia and prostatodynia.

### RELATED APPLICATIONS

This application is a continuation-in-part of U.S. patent application Ser. No. ______ for “ ” to Gerber et al. filed ______, U.S. patent application Ser. No. ______ for “ ” to Gerber et al. filed ______, U.S. patent application Ser. No. for “ ” to Gerber et al. filed and U.S. Provisional Patent Application Ser. No. 60/459,077 filed Mar. 31, 2003 entitled “ ” Method, System and Device for Treating Disorders of the Pelvic Floor by Means of Electrical Stimulation of the Pudendal and Associated Nerves, and the Optional Delivery of Drugs in Association Therewith” P-11358.00 to Spinelli et al., and claims priority and other benefits from each of the foregoing patent applications. This application hereby incorporates by reference herein the foregoing 'UUU, 'XXX, 'YYY, 'ZZZ patent applications, , each in its respective entirety.

### FIELD OF THE INVENTION

This invention relates to methods, systems and devices for treating various disorders of the pelvic floor by delivering electrical stimuli to the pudendal and sacral nerves.

### BACKGROUND

The medical device industry produces a wide variety of electronic and mechanical devices for treating patient medical conditions. Depending upon the medical condition, medical devices may be surgically implanted or connected externally to a patient receiving treatment. Clinicians use medical devices alone or in combination with drug therapies and surgery to treat patient medical conditions. For some medical conditions, medical devices provide the best (and sometimes the only) therapy to restore an individual to a more healthful condition and a fuller life. Conditions that medical devices may effectively treat include pelvic floor disorders.

Pelvic floor disorders adversely affect the health and quality of life of millions of people. Pelvic floor disorders include urinary control disorders such as urge incontinency, urge frequency, voiding efficiency, fecal control disorders, sexual dysfunction, and pelvic pain. Individuals with urinary control disorders often face debilitating challenges in their everyday lives. These individuals may be preoccupied with trips to the bathroom, fears of embarrassment and sleepless nights. Some suffers become so anxious that they become isolated and depressed. Pelvic floor disorders may be treated with a variety of therapeutic options such as behavior modification including biofeedback, pharmacological treatment, mechanical intervention such as self-catheterization, physical appliances such as diapers, and surgical intervention. Surgical treatments are the most invasive and are often considered after other therapies have proven ineffective.

Urinary incontinence, or the inability to control the passage of urine, is a relatively common problem. Although there are a variety of different types of urinary incontinence, stress incontinence, urge incontinence and urinary retention are the most common.

Stress incontinence is the unacceptable passage of urine under the stress of increased abdominal pressure. This increased pressure typically results from coughing, sneezing, and Valsalva. Stress incontinence is manifested urologically by normal cystometry, obtuse urethral vesicular angle, abnormally low urethral pressures and a physiologically short urethral length. This disorder is most common in multiparous, post-menopausal females. Physiologically, stress incontinence is a disorder of the volitional muscular control of the urethral resistance to the flow of urine. Laxity and partial denervation of the pelvic musculature is the chief abnormality.

Urge incontinence is the involuntary passage of urine with a concomitant sense of urgency. Systometry indicates detrusor (bladder wall muscle) contractions with low bladder filling pressures and volumes. These bladder contractions may not be inhibited in the presence of voluntary EMG signals from the sphincter, indicating reduction or loss of the pudendal-parasympathetic inhibitory reflex. Unsolicited, premature bladder contraction may result from mucosal irritation of varied etiology. These premature contractions of the bladder may also be the result of an abnormally high gain in the detrusor contractile reflex due to the loss of inhibitory control with an upper-motor-neuron lesion.

Urinary retention is characterized by the inability of a patient to spontaneously and controllably urinate or void. Catheterization of the urethra is provided to many patients suffering from urinary retention, which is often a painful and somewhat lengthy procedure having the added risk of causing infection.

Constipation is a life-disturbing problem that afflicts millions of Americans, from the very young to the elderly. Although relatively rare among the young, it is a very common problem in middle age, and is a nearly ubiquitous problem in the elderly. Chronic constipation is a major problem for many individuals, and frequently causes extreme discomfort to the afflicted. Such discomfort may be a major obstacle to leading a normal life, and may consume an enormous amount of the afflicted person's energy and time.

Besides causing severe discomfort, chronic constipation may also be harmful to the patient. For example, chronic constipation may result in an intestinal obstruction that may cause the patient great pain; or that may even cause the patient's death, unless surgically corrected. Chronic constipation may also prevent the patient from receiving the benefit of certain needed prescription medications, because the medications may have undesirable side effects on an already constipated gut.

Conventional therapies for chronic constipation are often distasteful and unpleasant at best, since they may involve treatments such as repeated consumption of large quantities of laxatives, repetitive use of enemas, or both. Repeatedly consuming large quantities of laxatives may be harmful to the patient, since they may result in dehydration or even renal failure. The repetitive use of enemas may be harmful since they may irritate or physically harm the treated portion of the patient's gut.

Chronic constipation is usually thought of in association with problems of the large intestine. Other parts of the patient's gut, however, may also exhibit chronic constipation-like problems, such as the esophagus, the stomach, and less frequently, the small intestine. Problems associated with chronic constipation may include depressed motility of the esophagus, stomach or small intestine. For simplicity, chronic constipation, or chronic constipation-like problems, of any portion of the patient's gut from the esophagus to the anus will be referred to hereafter as simply “constipation”.

The prostate is a glandular and fibromuscular organ in the male, which lies immediately below the bladder and surrounds the urethra. Prostatitis, the third leading disease of the prostate, is a common urologic condition that many clinicians find difficult to treat effectively.

The main symptom of chronic prostatitis (category III) is pain, followed by variable voiding (urgency/frequency) and erectile or sexual dysfunction. Patients have symptoms such as painful ejaculation or pain in the penis, testicles, or scrotum; low back, rectal or perineal pain; pain along the inner aspects of the thighs; irritative or obstructive urinary symptoms; and decreased libido or impotence. As a rule, chronic non-bacterial prostatitis patients do not have recurrent urinary tract infections.

Chronic prostatitis is a major male health issue. The average urologist in the U.S. sees 173 prostatitis patients per year, of which one-third are newly diagnosed. The prevalence of prostatitis in the general male population is estimated to be 5-8.8%, and it has been estimated that about 2 million office visits per year are related to prostatitis. Self-reported histories of prostatitis are as prevalent as 16% of all reported cases. Patients with chronic prostatitis experience a negative impact on quality of life comparable to patients with unstable angina, recent myocardial infarction or active Crohn's disease. The average age of the prostatitis population is estimated at 50 years. Prostatitis is the most common urologic diagnosis in men under 50 years old and the third most common in men over 50 years old. The most common classification of prostatitis is chronic prostatitis/chronic pelvic pain syndrome (category II), which may include as many as 90% of all patients who meet the criteria of the condition.

Despite the widespread prevalence of prostatitis, the diagnosis of chronic prostatitis represents a particular challenge since its diagnosis is often based on exclusion. Prostatitis remains poorly understood despite its prevalence because it encompasses multiple diverse disorders that cause symptoms related to the prostate gland. The etiology of acute and chronic bacterial prostatitis is clearly defined, and is a result of pathogenic bacteria that may cause systemic symptoms or urinary tract infections. On the other hand, chronic prostatitis/chronic pelvic pain syndrome does not have a clearly defined etiology, and there are many theories about the cause of this disease.

Perhaps the most comprehensive or encompassing theory of chronic non-bacterial prostatitis is one which advocates a multifactorial mechanism initiated by a stimulus such as infection or trauma. An interrelated cascade of events may follow, including physical, chemical, immunologic or neurogenic components, resulting in a local response of inflammation and/or neurogenic injury.

In the absence of consistent or clear etiologies for chronic prostatitis/chronic pelvic pain syndrome, improvement in quality of life and a reduction in symptoms are the usual goals of therapy. The most common treatment for chronic prostatitis involves pharmacologic treatments such as antibiotics, anti-inflammatory agents, alpha blockers, anti-spasmodics, analgesics, allopurinol, and muscle relaxants. Alpha blockers have successfully treated symptoms of prostatitis, although adverse event rates have been high. Muscle relaxants have shown significant improvement in small studies for category IIIB patients with sphincter dyssynergia or muscle spasm. Anti-inflammatory agents, such as pentosan polysulfate, have proven successful for approximately 40% of patients with category IIIA prostatitis.

Phytotherapeutic agents have demonstrated improvements in small studies for pain and irritative voiding. Other treatments include physiotherapy (such as biofeedback and pelvic muscle exercises) and various modalities of invasive and minimally invasive procedures (e.g., transurethral microwave therapy, transurethral incision of the bladder neck, hydrodistensions, acupuncture, electroneuromodulation, balloon dilation, YAG laser therapy and heat therapy). Repetitive prostatic massage is a popular treatment method due to the failure of consistent standard medical therapy to treat the condition. Lifestyle changes, such as meditation, discontinuation of bike riding, sitz-baths, dietary changes and chiropractic therapy, are often prescribed.

As a result of unknown etiology, unsure diagnosis and treatment options that are often myriad and ineffective, chronic prostatitis is a “diagnosis of exclusion” and has a poor record of treatment success. Accordingly, the present invention is intended to provide solutions to the foregoing problems through improved and more effective methods of treating pain and other symptoms associated with chronic prostatitis, prostatalgia and prostatodynia.

Sexual dysfunction comprises a broad range of maladies, including erectile dysfunction, orgasmic dysfunction, premature ejaculation and lack of lubrication. Sexual dysfunctions plague both women and men, and may be life-long or acquired. To treat impotence (also called erectile dysfunction), electrical conductors may be implanted near the surface of the pelvic splanchnic nerve. Stimulation of this nerve with low voltage electrical pulses is believed to cause arterioles dilation and initiate erection. Also, it is known that implantation of an electrode on the cavernous nerves of a male, adjacent to his prostate gland, may also cause penile erection. Further, other electrical impulse devices exist that are not implanted but instead apply electrical stimuli topically to the coccyx region to promote sexual excitation. Impotence, however, should not be confused with orgasmic dysfunction, where satisfactory erection may be obtained but there is an absence of orgasm.

Current treatment of orgasmic dysfunction concentrates on the psychological components of the disorder rather than the physiological components. Orgasmic dysfunction is a physical malady that results in marked distress and interpersonal difficulty. The physical disorder causes psychological performance anxiety and pressure. Sexual desire and frequency usually decline. The patient's intimate relationships usually suffer from resentment and conflict. There is anecdotal evidence of patients who have experienced mild sensations in the genitalia while undergoing spinal cord stimulation for pain relief.

Spinal cord stimulation, on the other hand, has been used as a treatment for chronic painful conditions for approximately thirty years. Commonly, spinal cord stimulation is used to alleviate pain after failed surgery, pain due to neuropathies, or pain due to inadequate blood flow. Neurostimulation systems have been found to relieve chronic, intractable pain in the limbs or trunk.

The basic concept of neurostimulation as it relates to pain relief involves the substitution of sensations that reach the thalamus of the brain. Rather than a pain message, the spinal cord stimulation closes the gate in the spinal cord and replaces the pain sensation with a tingling sensation. Electrodes are positioned effectively to create paresthesia in the painful area. Paresthesia refers to a change in sensation in an area of the body. Usually paresthesia is used to show change in neurologic function caused by damage to a nerve or nerves. Paresthesia is usually not an absence of sensation, but a decrease or alteration of sensation. Patients have described paresthesia as a “buzzing sensation.”

Paresthesia is accomplished through the implantation of stimulating electrodes within or near the spinal cord. The electrodes are inserted between the vertebrae in parallel with the spinal cord. Low-voltage electrical stimulation is precisely applied to the spinal cord. Through direct stimulation of the dorsal column or the targeted peripheral nerve, the sensation of pain is replaced by a more pleasant “tingling” sensation. The sensation may be adjusted in terms of amplitude to control intensity and pulse width to control duration and frequency. Usually such neurostimulation systems are implantable. Medtronic Neurological, a division of Medtronic, Inc. of Minneapolis, Minn., sells a neurostimulator system used for pain relief. The device has been approved by the Federal Drug Administration for implantation in the spinal cord to alleviate pain.

One surgical technique to treat urinary control disorders is the implantable InterStim® therapy, available from Medtronic, Inc., which applies mild electrical stimulation to the sacral nerves in the lower region of the spine to influence the behavior of structures such as the bladder, sphincter and pelvic floor muscles. Generally, implantation of the InterStim system involves surgically implanting a stimulation lead near the sacral nerves. The stimulation lead is a very small, insulated, electrical conductor with electrical stimulation contacts on the distal end for implantation near the sacral nerves and an electrical connector on the proximal end of the lead. The lead electrical connector is typically connected to a small extension, and the extension is connected to a small neurostimulator that operates in a fashion broadly similar to that of a cardiac pacemaker by delivering occasional small electrical pulses that sometimes create a tingling sensation felt by the patient. The stimulation lead, lead extension and neurostimulator are all implanted in the patient in a manner that is typically not perceptible by others. InterStim therapy may improve the condition of a pelvic floor disorder patient and allow the patient to lead a full life. InterStim therapy is also nondestructive and reversible.

Each year thousands of patients have sacral nerve stimulation systems implanted within them for the treatment of urinary incontinence and urinary retention. Therapy success is determined through the evaluation of symptoms related to the disorder. Clinical success for most therapies, including sacral nerve stimulation, is defined as a 50% decrease in the following symptoms:

Urge incontinence as measured by:
Average number of incontinent episodes per day, or
Average severity ranking of incontinent episodes, or
Average number of absorbent pads or diapers replaced due to incontinence.

Urinary frequency and urgency as measured by:
Average number of voids per day, or
Average voided volume per void, or
Average degree of urgency prior to voiding.

Urinary retention as measured by:
Decrease in post-void urine residual, or
Average number of catheterizations consisting of ≧100 ml of urine, or
Average catheter volume per catheterisation (post-void residual).

Today, electrical stimulation of the sacral nerve is fairly common for the purpose of treating voiding dysfunction. Although the majority of patients receiving sacral nerve stimulation obtain satisfactory relief of their voiding dysfunction, some patients (less than 50%) do not experience adequate relief from sacral nerve stimulation techniques or desire to obtain better results from the therapy.

Electrical stimulation delivered by an intravaginal or a perineal surface electrode has been shown to inhibit premature and inappropriate detrusor contractions. The mechanism for such effects appears to derive from the electrical stimulation of pudendal nerve afferents (sensory receptors or sensory nerve fibers). Input into the pudendal afferent system inhibits a parasympathetic reflex loop consisting of bladder wall afferents (sensory reflexes) and efferents (motor reflexes). This parasympathetic loop normally senses a distension of the bladder via the afferent limb and responds by sending an efferent signal to contract the bladder. Although such stimulation has shown therapeutic effects, electrode placement and on-going stimulation do not lend themselves easily to chronic stimulation.

Stimulation of the pudendal nerve as an alternative to sacral nerve stimulation has long been proposed. Until the recent development of new stimulation lead technology, the invasiveness of the surgical procedure made stimulation of the pudendal nerve impractical. Since the pudendal nerve directly innervates much of the pelvic floor, it is believed to be a more optimal stimulation site with few undesired side effects. Advancements in minimally invasive lead placement techniques along with advancement in lead anchoring techniques have resulted in the increased viability of chronic stimulation of the pudendal nerve.

Some prior art publications relating to various embodiments of the present invention are listed in Table 1 below.

TABLE 1
Prior Art Publications

Juenemann et al., “Clinical Signifimayce of Sacral and Pudendal Nerve
Anatomy,” The Journal of Urology, Vol. 139, pp. 74-80 (January,
1988).
Schmidt, Richard A., “Technique of Pudendal Nerve Localization for
Block or Stimulation,” The Journal of Urology,
Vol. 142(December, 1989).
U.S. Pat. No. 4,406,288 to Cash for “Bladder Control Device and
Method”
U.S. Pat. No. 4,607,639 to Tanagho et al. for “Method and System for
Controlling Bladder Evacuation.”
U.S. Pat. No. 4,771,779 to Tanagho et al. for “System for Controlling
Bladder Evacuation.”
U.S. Pat. No. 4,739,764 to Lue et al. for “Method for Stimulating Pelvic
Floor Muscles for Regulating Pelvic Viscera.”
U.S. Pat. No. 4,881,526 to Johnson et al. for “Intravaginal Electrode and
Stimulation System for Controlling Female Urinary Incontinence”
U.S. Pat. No. 5,425,751 to Baeten et al. for “Method and Apparatus for
Optimum Positioning of a Muscle Stimulating Implant”
U.S. Pat. No. 5,540,730 to Terry, Jr. et al. for “Treatment of Motility
Disorders by Nerve Stimulation”
U.S. Pat. No. 5,984,854 to Ishikawa et al. for “Method for Treating
Urinary Incontinence and Apparatus Therefor”
U.S. Pat. No. 6,055,456 to Gerber for “Single and Multi-Polar
Implantable Lead for Sacral Nerve Stimulation”
U.S. Pat. No. 6,366,814 to Boveja. for “Electrical Stimulation Adjunct
(Add-On) Therapy for Urinary Incontinence and Urological
Disorders Using an External Stimulator”
U.S. Pat. No. 6,449,512 to Boveja. for “Apparatus and Method for
Treatment of Urological Disorders Using Programmerless
Implantable Pulse Generator System”
U.S. Pat. No. 6,587,719 to Barrett et al. for “Treatment of Obesity by
Bilateral Vagus Nerve Stimulation”
U.S. Pat. No. 6,609,025 to Barrett et al. for “Treatment of Obesity by
Bilateral Sub-Diaphragmatic Nerve Stimulation”
U.S. Patent Application Publication No. 2002/0055761 to Mann et al. for
“Implantable Stimulator Systems and Methods for Treatment of
Incontinence and Pain”
U.S. Patent Application Publication No. 2002/0055779 to Andrews for
“Neural Prosthesis”
PCT Patent Application WO 02/078592 to Grill et al. for “Systems and
Methods for Selectively Stimulating Components In, On or Near the
Pudendal Nerve or Its Branches to Achieve Selective Physiologic
Responses”
European Patent Application No. 0 245 547 to Tanagho et al. for
“Electronic Control System for Controlling Pelvic Viscera via Neuro-
Electrical Stimulation.”

All patents and technical papers listed in Table 1 hereinabove are hereby incorporated by reference herein, each in its respective entirety. As those of ordinary skill in the art will appreciate readily upon reading the Summary of the Invention, Detailed Description of the Preferred Embodiments and Claims set forth below, at least some of the devices and methods disclosed in the patents and publications of Table 1 may be modified advantageously in accordance with the teachings of the present invention. The foregoing and other objects, features and advantages, which will now become more readily apparent by referring to the following specification, drawings and claims, are provided by the various embodiments of the present invention.

### SUMMARY OF THE INVENTION

Simultaneous or concurrent electrical stimulation of the pudendal and sacral nerves has been discovered to provide a means of more directly or effectively stimulating portions of the pelvic floor than has been possible heretofore employing conventional sacral or pudendal nerve electrical stimulation techniques. It is believed that the pudendal nerve more directly innervates the pelvic floor and portions thereof than does stimulation of a sacral nerve. It has been discovered that electrical stimulation of the pudendal nerve or portions thereof provides beneficial effects and therapy for various disorders of the pelvic floor over a wider anatomical region than merely the pudendal nerve or portion thereof which is being stimulated, or than may be attained through conventional sacral nerve stimulation. Because the present invention provides for more targeted electrical stimulation of the pelvic floor or portions thereof, at least some of the undesirable side effects of sacral nerve stimulation may be avoided or minimized.

One or more electrical stimulation signals are applied, and optionally one or more drugs are infused, injected or otherwise administered, to appropriate portions of a patient's pelvic floor and pudendal nerve or portions thereof in an amount and manner effective to treat a number of disorders, including, but not limited to, urinary and/or fecal voiding dysfunctions such as constipation, incontinence disorders such as urge frequency and urinary retention disorders, sexual dysfunctions such as orgasmic and erectile dysfunction, pelvic pain, prostatitis, prostatalgia and prostatodynia.

The at least one electrical stimulation signal is applied by an IMD that has at least one medical electrical lead positionable, secured or attached to or in a patient's pelvic floor and in proximity to a pudendal nerve or portion thereof. Each such lead carries at least one electrode, and preferably at least two electrodes, positionable or attachable for contact with or in proximity to the patient's pudendal nerve or portion thereof.

Various embodiments of the present invention are capable of providing one or more solutions to one or more problems existing in the prior art respecting conventional treatment for pelvic pain, sexual dysfunction, prostadynia, prostatitis, prostatalgia, and/or urinary or fecal incontinence in a patient. Such problems include, but are not limited to, one or more of: (a) sequelae or side-effects resulting from the administration of pharmaceutical products; (b) the requirement to purchase expensive pharmaceutical products on an on-going basis; (c) not having the ability to terminate or change instantaneously administration of pharmaceutical therapy; (d) not having the ability to target with a great deal of precision or specificity the ailment in question using pharmaceutical products; (e) in the case of electrical stimulation, not having a well-defined or reliable method of determining stimulation electrode placement; (f) patients having chronic and essentially untreatable pain having no effective pain relief therapy available for use; (g) patients having to wear diapers, pads or other devices for containing human waste, and/or (f) conventional sacral nerve stimulation techniques being incapable of providing the desired relief or therapy in many patients.

Various embodiments of the present invention are capable of providing one or more advantages, which may include, but are not necessarily limited to: (a) determining with a high degree of precision the optimal location for one or more stimulation electrodes in a patient; (b) with a chronic stimulation lead, providing a relatively reliable method of replicating the electrode position of a screening or temporary stimulation lead when implanting the chronic lead in a patient; (c) determining with a high degree of confidence, before or during the implantation procedure, whether electrical stimulation techniques are capable of providing the desired relief or therapy to a patient; (d) in accordance with (c), preventing unnecessary implants of electrical stimulation devices in patients; (e) targeting delivery of therapy with a high degree of specificity; (f) having the ability to change the therapy delivered on-demand or instantaneously; (g) lowering medical care costs in respect of pharmaceutical products; (h) having the potential to delivery superior therapy; (i) a patient not having to remember to take a drug daily or according to a predetermined regimen; (j) permitting stimulation lead implantation surgical procedures to be completed more quickly; (d) reducing trauma or damage to a patient's pelvic floor anatomy; and/or (e) improved physical and electrical coupling of one or more stimulation electrodes to a pertinent nerve or nerve portion.

### BRIEF DESCRIPTIONS OF THE DRAWINGS

These and other objects, features and advantages of the present invention will be more readily understood from the following detailed description of the preferred embodiments thereof, when considered in conjunction with the drawings, in which like reference numerals indicate identical structures throughout the several views when appropriate. Note that the drawings are not necessarily to scale.

FIG. 1 shows one embodiment of the present invention, where INS 10 is implanted in an upper buttock position in a patient, lead 16 is implanted near or adjacent to sacral nerve or nerve portion 25, and lead 18 is implanted near or adjacent to pudendal nerve or nerve portion 26, to thereby effect therapeutic relief;

FIG. 2 shows a block diagram illustrating some of the constituent components of INS 10 in accordance with one embodiment of the present invention;

FIG. 3 shows a simplified anatomical view of the pelvic floor of a human patient, the locations of the pudendal, sacral and associated nerves therein, and an illustrative positioning of IMD 10, electrical stimulation leads 16 and 18, and corresponding electrodes 20-23 and 40-43;

FIG. 4 shows a different simplified anatomical view of the pelvic floor of a human patient, the locations of the pudendal, sacral and associated nerves therein, and a different illustrative positioning of IMD 10, electrical stimulation leads 16 and 18, and corresponding electrodes 20-23 and 40-43;

FIG. 5 shows a simplified male anatomical view of the pelvic floor and the locations of the pudendal, sacral and associated nerves therein;

FIGS. 6A through 6E show various embodiments of the distal end of leads 16 and 18 of the present invention;

FIG. 7 shows a flow diagram according to one embodiment of a method of the present invention for stimulating the pudendal and sacral nerves, and

FIGS. 8A through 8F shows various embodiments of the first and second pulse regimes of the present invention.

### DETAILED DESCRIPTIONS OF THE PREFERRED EMBODIMENTS

In the following description of the preferred embodiments, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims. As employed herein, the term “sacral nerve 25” means any one of the sacral nerves, portions of the sacral nerve(s), nerves neurologically connected to any one of the sacral nerves and in relatively close physical proximity thereto, and extensions or branches of any one of the sacral nerves. As employed herein, the term “pudendal nerve 26” means the pudendal nerve itself, portions of the pudendal nerve, nerves neurologically connected to the pudendal nerve and in relatively close physical proximity thereto, and extensions or branches of the pudendal nerve.

Augmentation of the sacral nerve stimulation with pudendal nerve stimulation, the broader, yet more specific stimulation may help the patient achieve better clinical outcomes. The nerve integrity of some patients may be compromised due to the progression of a neurological disease such as multiple sclerosis or Parkinson's disease. Other patients may have compromised nerves due to injury caused by obstetrics or accidents. In the case of a compromised neurological system in the pelvic floor, signal conduction may be a major issue and a factor in their incontinence. Because the pelvic floor is innervated by nerve fibers from each of the sacral nerves, stimulation of a single sacral nerve does not always give adequate or full relief of the patient's incontinence.

Stimulation of both the sacral nerve and the pudendal nerve will provide a broader stimulation pattern covering more of the pelvic floor and may result in additional relief of the incontinence symptoms. In addition to the sacral nerves, the pudendal nerve is a good site to stimulate because it is innervates much of the pelvic floor including the urinary sphincters.

The sacral nerves innervate the pelvic floor and the legs and feet. Stimulation of the sacral nerve results in stimulation of both the pelvic floor as well as the leg and foot. One issue that many sacral nerve stimulation patients experience is an annoying stimulation of the leg and/or foot. This issue may many times be mitigated through reducing the stimulation level on the sacral nerve. An advantage to stimulating both the sacral nerve and the pudendal nerve is that lower stimulation levels may be used to achieve the same or better therapeutic results which may eliminate the annoying stimulation of the leg and/or foot. The lower stimulation levels will result in less sensory stimulation and will result in the patient being less aware of the presence of the stimulation. An additional advantage is that the lower stimulation levels will result in more longevity from the implanted pulse generator.

The innervation of the sacral nerves and the pudendal nerves is lateralized. The stimulation of the pudendal sacral nerve and stimulation of the pudendal nerve may occur on opposite sides. For example, the sacral nerve may be stimulated on the left side while the pudendal nerve may be stimulated on the right side. Nerve laterilization selection is determined through factors such as nerve EMG response, anatomical access, physician preference and patient preference.

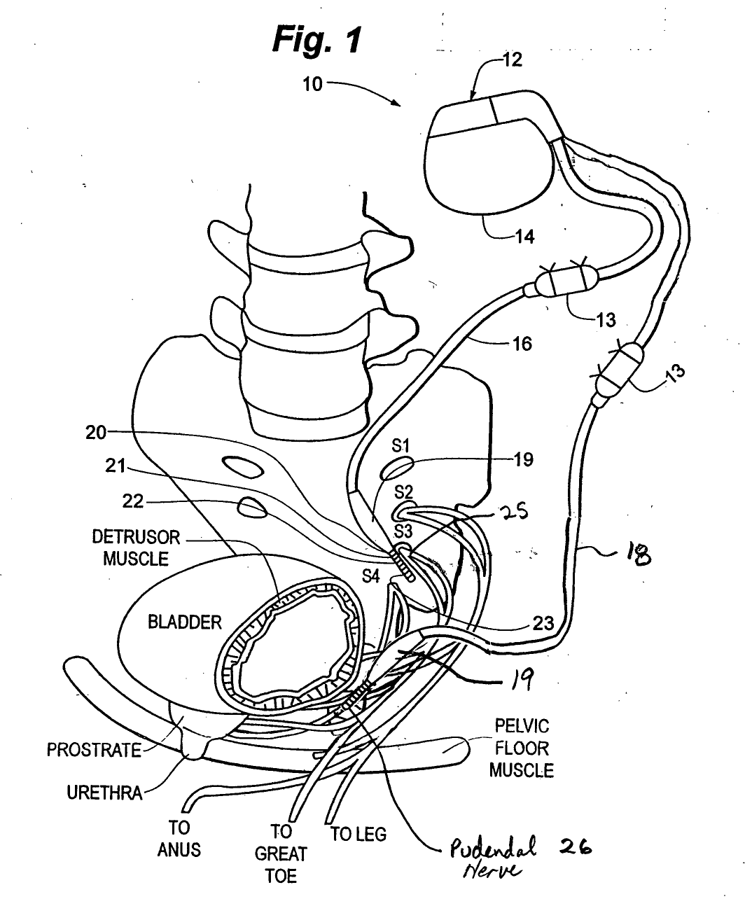

FIG. 1 shows one embodiment of the present invention, where INS 10 is implanted in an upper buttock position in a patient. Hermetically sealed enclosure 14 is preferably formed of a biocompatible material such as an appropriate metal alloy containing titanium. In FIG. 1, lead 16 is implanted near or adjacent to sacral nerve or nerve portion 25 (such as the S3 sacral nerve), and lead 18 is implanted near or adjacent to pudendal nerve or nerve portion 26 to thereby effect therapeutic relief. Note, however, that INS 10 may be implanted in any appropriate location in the patient, such as in the abdomen or side.

Relief is effected by INS 10 and leads 16 and 18 as a result of electrical stimulation signals being delivered to or near sacral nerve 25 or nerve portion 8 by electrodes 20-23, and to or near pudendal nerve 26 or nerve portion 8 by electrodes 20-23. One, two, three, four or more electrodes 20, 21, 22 and 23 may be disposed at the distal end of lead 16. FIG. 1 shows four electrodes located at the distal end of lead 16 near pudendal nerve 26 and additionally near sacral nerve 25. Other lead locations, electrode configurations and lead configurations are possible and contemplated in the present invention.

In one embodiment of the present invention, lead 16 and/or 18 provides electrical stimulation pulses to the desired nerve target sites or portions 25 and/or 26 and thereby stimulates the target nerve or nerve portion located in the vicinity of the electrode(s) thereof. Lead 16 and/or 18 may have unipolar electrodes disposed thereon (where enclosure 14 is employed as an indifferent electrode) or may have bipolar electrodes disposed thereon, where one or more electrodes disposed on a lead are employed as the indifferent electrode. In one embodiment of the present invention, Lead 16 and/or 18 extends from lead connector 13, which in turn forms an integral portion of lead extension 15 connected at its proximal end to connector header module 12.

Typically, leads 16 and 18 are tunneled subcutaneously between the location of INS 10 and the location or site of the nerve or nerve portion 25 and/or 26 that is to be stimulated. INS 10 is typically implanted in a subcutaneous pocket formed beneath the patient's skin according to methods well known in the art. Further details concerning various methods of implanting INS 10 and leads 16 and 18 are disclosed in the Medtronic Interstim Therapy Reference Guide published in 1999, the entirety of which is hereby incorporated by reference herein. Other known methods of implanting and locating leads 16 and 18 are of course contemplated in the present invention.

Some representative examples of leads 16 and 18 include MEDTRONIC nerve stimulation lead model numbers 3080, 3086, 3092, 3487, 3966 and 4350 as described in the MEDTRONIC Instruction for Use Manuals thereof, all hereby incorporated by reference herein, each in its respective entirety. Some representative examples of INS 10 include MEDTRONIC implantable electrical stimulator model numbers 3023, 7424, 7425 and 7427 as described in the Instructions for Use Manuals thereof, all hereby incorporated by reference herein, each in its respective entirety. INS 10 may also be constructed or operate in accordance with at least some portions of the implantable stimulators disclosed in U.S. Pat. No. 5,199,428 to Obel et al., U.S. Pat. No. 5,207,218 to Carpentier et al. or U.S. Pat. No. 5,330,507 to Schwartz, all of which are hereby incorporated by reference herein, each in its respective entirety.

U.S. patent application Ser. No. 10/004,732 entitled “Implantable Medical Electrical Stimulation Lead Fixation Method and Apparatus” and U.S. patent application Ser. No. 09/713,598 entitled “Minimally Invasive Apparatus for Implanting a Sacral Stimulation Lead ” to Mamo et al., the respective entireties of which are hereby incorporated by reference herein, describe methods of percutaneously introducing lead 16 and/or 18 to a desired nerve stimulation site in a patient.

Certain aspects of the subject matter described in U.S. Provisional patent application Ser. No. 60/459,077 entitled “Method, System and Device for Treating Disorders of the Pelvic Floor by means of Electrical Stimulation of the Pudendal and Associated Nerves, and the Optional Delivery of Drugs in association”, where various methods of positioning and implanting a medical electrical lead 16 and/or 18 so as to provide optimal stimulation of the pudendal nerve 26 or a portion thereof, may be adapted for use in conjunction with at least some embodiments of the present invention.

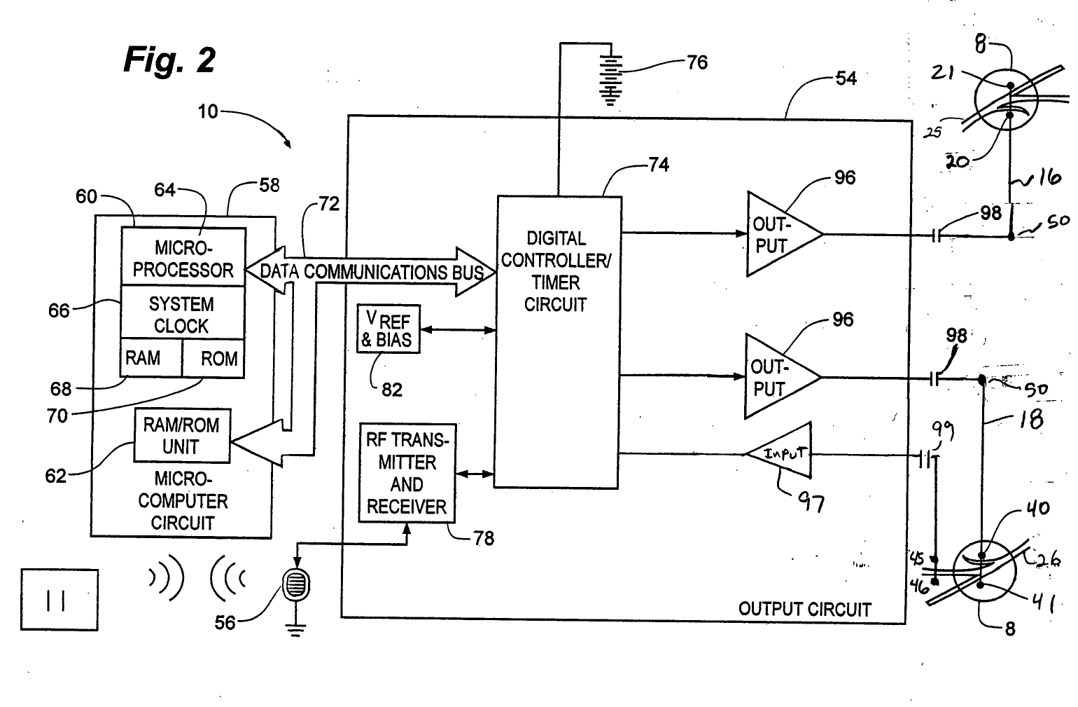

FIG. 2 shows a block diagram illustrating some of the constituent components of INS 10 in accordance with one embodiment of the present invention, where INS 10 is an implantable electrical stimulator having a microprocessor- or controller-based architecture. Other architectures of INS 10 are of course contemplated in the present invention, such as the logic or state machine architecture employed in the Medtronic Model Number 3023 INS. For the sake of convenience, INS 10 in FIG. 2 is shown with two leads 16 and 18 connected thereto; similar circuitry and connections not shown in FIG. 2 apply generally to other possible additional leads not shown in the drawings.

The system comprising INS 10 and leads 16 and 18 may be an open-loop non-feedback-control system, or a closed-loop feedback control system. In the case of a closed-loop feedback control embodiment of the present invention, FIG. 2 shows optional input amplifier 97 connected to sensing electrodes 45 and 46 through capacitor 99.

INS 10 in FIG. 2 is most preferably programmable by means of external programming unit 11. One such programmer is the commercially available Medtronic Model No. 7432 programmer, which is microprocessor-based and provides a series of encoded signals to INS 10, typically through a programming head which transmits or telemeters radio-frequency (RF) encoded signals to INS 10. Another suitable programmer is the commercially available Medtronic Model No. 8840 programmer, which is also microprocessor-based but features a touch control screen. Any of a number of suitable programming and telemetry methodologies known in the art may be employed so long as the desired information is transmitted to and from the implantable electrical stimulator 10.

FIG. 2 further shows a block diagram illustrating some of the constituent components of INS 10 in accordance with one embodiment of the present invention. Leads 16 and 18 are coupled to nodes 50 in INS 10 through capacitor 98. Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and ROM 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 2 are powered by an appropriate implantable primary (i.e., non-rechargeable) battery power source 76 or secondary (i.e., rechargeable) battery power source 76. For the sake of clarity, the coupling of battery 76 to the various components of INS 10 is not shown in FIG. 2. Antenna 56 is connected to microcomputer circuit 58 via digital controller/timer circuit 74 and data communication bus 72 to permit uplink/downlink telemetry through RF transmitter and receiver telemetry unit 78. By way of example, telemetry unit 78 may correspond to that disclosed in U.S. Pat. No. 4,566,063 issued to Thompson et al. It is generally preferred that the particular programming and telemetry scheme selected permit the entry and storage of electrical stimulation parameters. The specific embodiments of antenna 56 and other telemetry circuitry presented herein are shown for illustrative purposes only, and are not intended to limit the scope of the present invention.

Continuing to refer to FIG. 2, V_{REF} and bias circuit 82 most preferably generate stable voltage reference and bias currents for analog circuits included in output circuit 54. Operating commands for controlling the timing of INS 10 are coupled by data bus 72 to digital controller/timer circuit 74, where digital timers and counters establish the specific stimulation parameters of INS 10 as well as various timing windows for controlling the operation of peripheral components disposed within input/output circuit 54. Output pulse generator 96 provides electrical stimuli to desired nerve and/or nerve portion 25 or 26 through coupling capacitor 98 in response to a trigger signal provided by digital controller/timer circuit 74, when an externally transmitted stimulation command is received, or when a response to other stored commands is received.

By way of example, output amplifier 96 may correspond generally to an output amplifier disclosed in U.S. Pat. No. 4,476,868 to Thompson, hereby incorporated by reference herein in its entirety. The specific embodiments of output amplifier 96 identified herein are presented for illustrative purposes only, and are not intended to be limiting in respect of the scope of the present invention. The specific embodiments of such circuits may not be critical to practicing some embodiments of the present invention so long as they provide means for generating an appropriate train of stimulating pulses to desired nerve or nerve portion 25 and/or 26.

In various embodiments of the present invention, INS 10 may be programmably configured to operate so that it varies the rate at which it delivers stimulating pulses to desired nerve or nerve portion 25 and/or 26 in response to one or more selected outputs being generated. INS 10 may further be programmably configured to operate so that it may vary the morphology of the stimulating pulses it delivers. Numerous implantable electrical stimulator features and functions not explicitly mentioned herein may be incorporated into INS 10 while remaining within the scope of the present invention. Various embodiments of the present invention may be practiced in conjunction with one, two, three or more leads, or in conjunction with one, two, three, four or more electrodes disposed on each lead.

Leadless embodiments of the present invention are also contemplated, where one or more stimulation and/or sensing electrode capsules or modules are implanted at or near a desired nerve or nerve portion 25 and/or 26, and the capsules or modules deliver electrical stimuli directly to the selected site using a preprogrammed stimulation regime, and/or the capsules or modules sense electrical or other pertinent signals. Such capsules or modules are preferably powered by rechargeable batteries that may be recharged by an external battery charger using well-known inductive coil or antenna recharging means, and preferably contain electronic circuitry sufficient to permit telemetric communication with a programmer, to deliver electrical stimuli and/or sense electrical or other signals, and to store and execute instructions or data received from the programmer. Alternatively, in one embodiment of the present invention INS 10 is configured to recharge such a remotely positioned capsule or module by RF means on a periodic basis according to battery state of charge requirements measured or exhibited by such remote capsule or module.

Examples of methods and devices that may be adapted for use in the wireless devices and methods of the present invention include those described in U.S. Pat. No. 6,208,894 to Schulman et al. entitled “System of implantable devices for monitoring and/or affecting body parameters;” U.S. Pat. No. 5,876,425 to Schulman et al. entitled “Power control loop for implantable tissue stimulator;” U.S. Pat. No. 5,957,958 to Schulman et al. entitled “Implantable electrode arrays;” U.S. patent application Ser. No. 09/030,106 filed Feb. 25, 1998 to Schulman et al. entitled “Battery-Powered Patient Implantable Device,” and U.S. Pat. No. 6,650,943 to Whitehurst et al. entitled “Fully Implantable Neurostimulator for Cavernous Nerve Stimulation as a Therapy for Erectile Dysfunction and Other Sexual Dysfunction.”

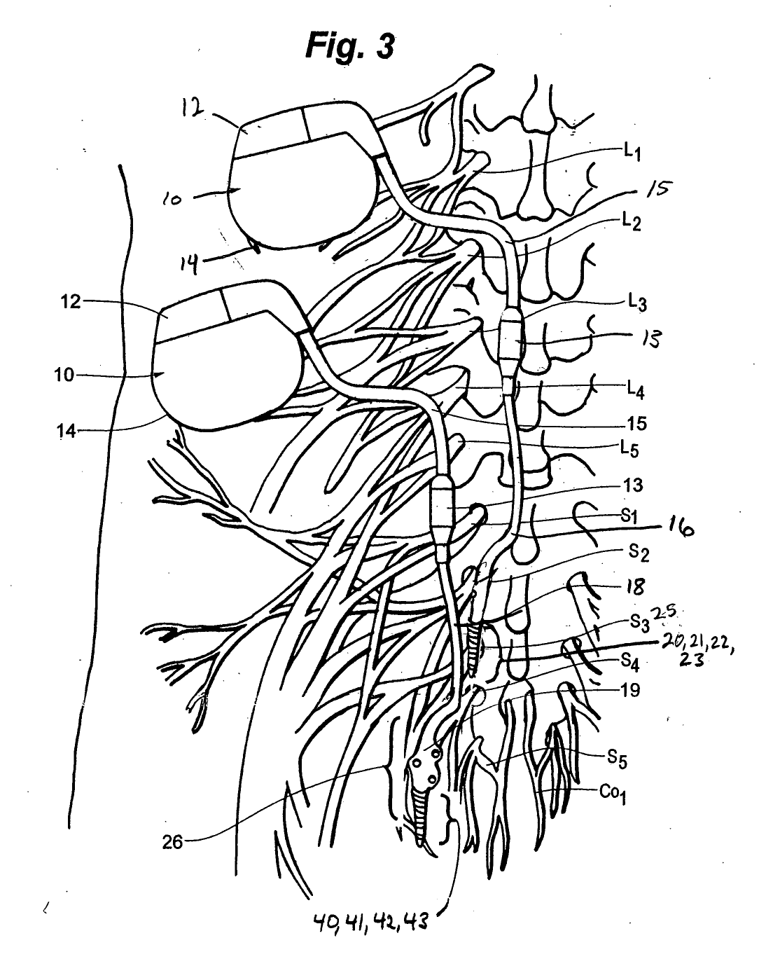

FIG. 3 shows a simplified anatomical view of the pelvic floor of a human patient, the locations of pudendal nerves 26, sacral nerves 25, and other associated nerves and nerve portions therein, as well as illustrative positions for IMD 10, electrical stimulation leads 16 and 18, and corresponding electrodes 20-23 and 40-43. FIG. 3 shows INS 10 implanted in an appropriate location within the patient, with lead 16 being implanted near or adjacent to one or more of sacral nerves S1, S2, S3 25 and S4, and lead 18 implanted near or adjacent to pudendal nerve or nerve portion 26 to thereby effect therapeutic relief. Such relief is effected as a result of electrical stimulation signals being delivered to or near to or near one or more of such nerves S1, S2, S3 25, S4, and/or pudendal nerve or nerve portion 26, or a nerve(s) in proximity thereto, by electrodes 20, 21, 22, and/or 23 and electrodes 40, 41, 42 and/or 44. One, two, three, four or more electrodes 20, 21, 22 and 23 may be disposed at the distal end of lead 16. One, two, three, four or more electrodes 40, 41, 42 and 43 may be disposed at the distal end of lead 18. Consistent with the foregoing description, other lead locations and electrode configurations are of course possible and contemplated in the present invention.

FIG. 4 shows a different simplified anatomical view of the pelvic floor of a human patient, the locations of pudendal nerve or nerve portion 26, sacral nerve or nerve portion 25, and other associated nerves and nerve portions therein, as well as illustrative positions for IMD 10, electrical stimulation leads 16 and 18, and corresponding electrodes 20-23 and 40-43. As shown in FIG. 4, pudendal nerve or 26 innervates the pelvic floor muscle and sphincters. FIG. 4 shows INS 10 implanted in an appropriate location within the patient, with lead 16 being implanted near or adjacent to one or more of sacral nerves S1, S2, S3 25 and S4, and lead 18 implanted near or adjacent to pudendal nerve or nerve portion 26 to thereby effect therapeutic relief. Such relief is effected as a result of electrical stimulation signals being delivered to or near to or near one or more of such nerves S1, S2, S3 25, S4, and/or pudendal nerve or nerve portion 26, or a nerve(s) in proximity thereto, by electrodes 20, 21, 22, and/or 23 and electrodes 40, 41, 42 and/or 44. One, two, three, four or more electrodes 20, 21, 22 and 23 may be disposed at the distal end of lead 16. One, two, three, four or more electrodes 40, 41, 42 and 43 may be disposed at the distal end of lead 18. Consistent with the foregoing description, other lead locations and electrode configurations are of course possible and contemplated in the present invention.

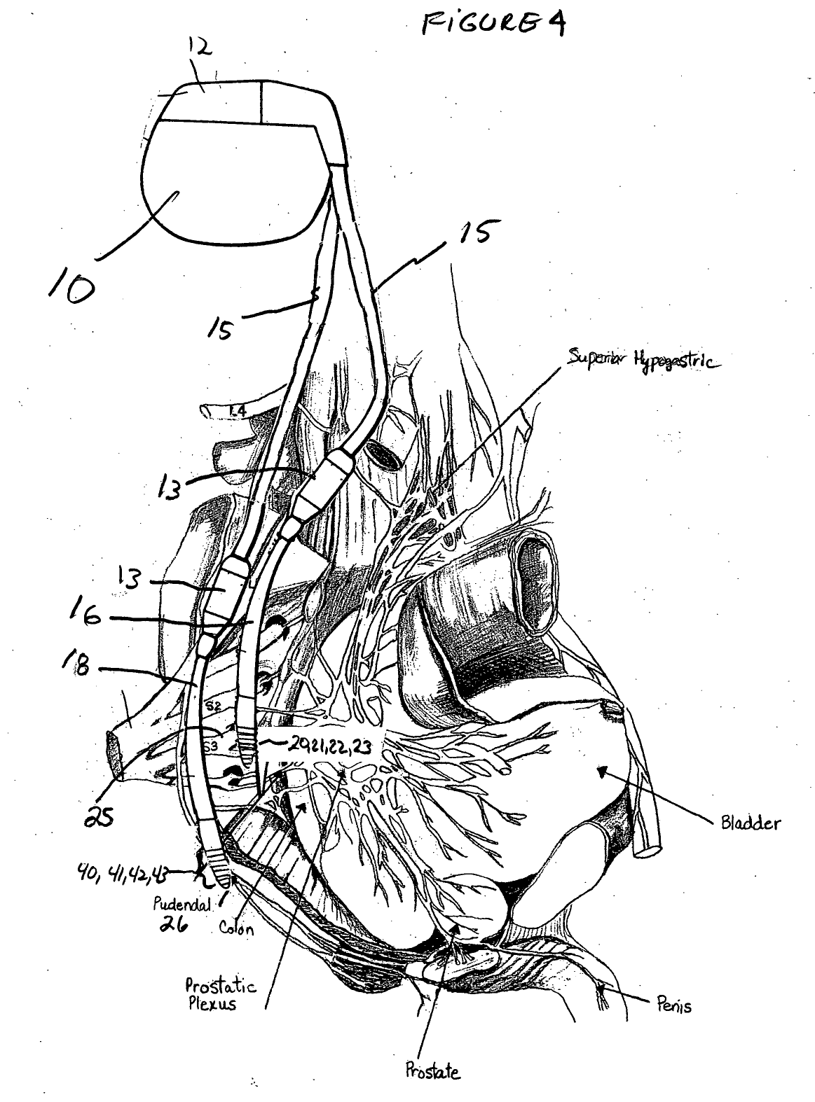

FIG. 5 shows a simplified male anatomical view of the pelvic floor and the locations of pudendal nerve 26 and nerves associated therewith, where in accordance with some embodiments of the present invention leads 16 and/or 18 and electrodes 20-23 and/or 40-43 may be attached, connected or implanted in proximity thereto. Pudendal nerve 26 may be seen to extend downwardly past sacrospinal ligament 43, greater sciatic foramen 48, and lesser sciatic foramen 49, and thereafter to branch into inferior rectal nerves 51, perineal nerves 52, scrotal nerves 53 and dorsal nerve 55 of penis 35.

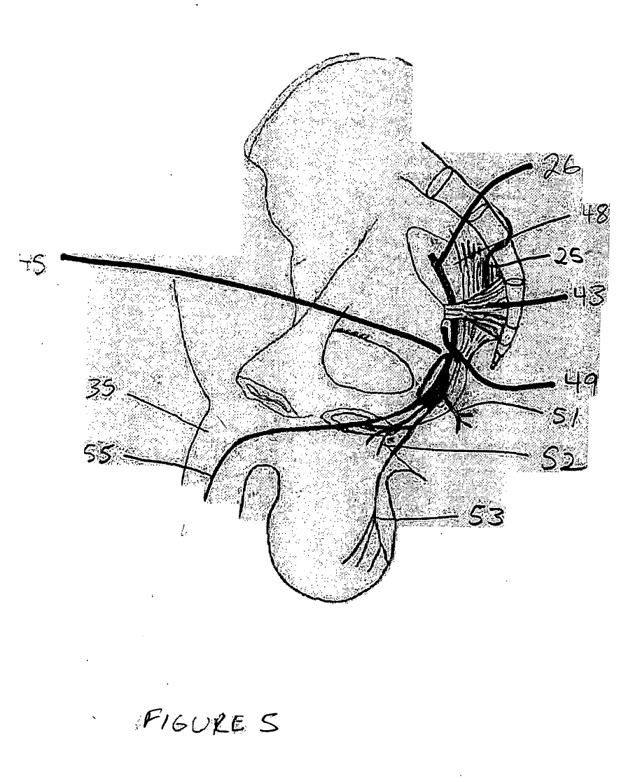

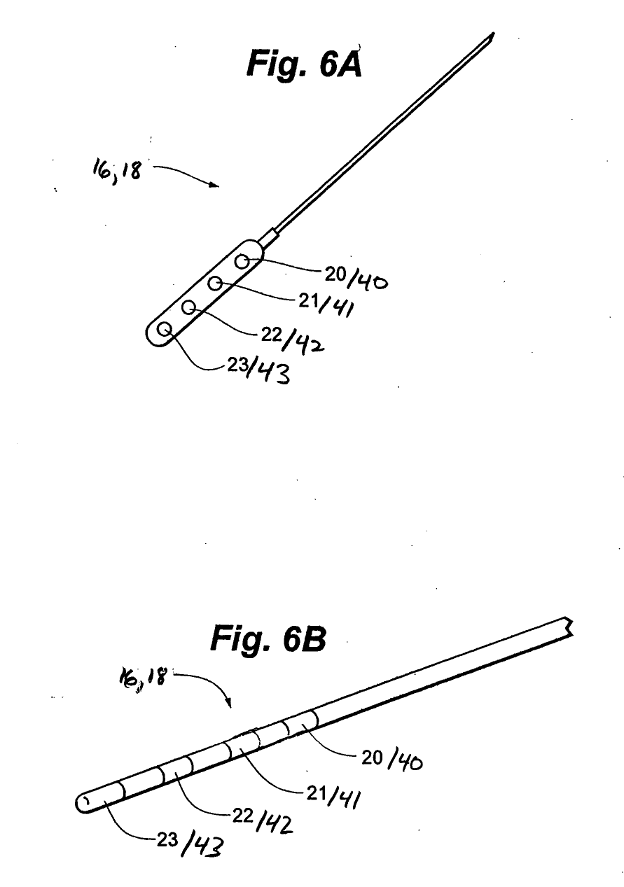

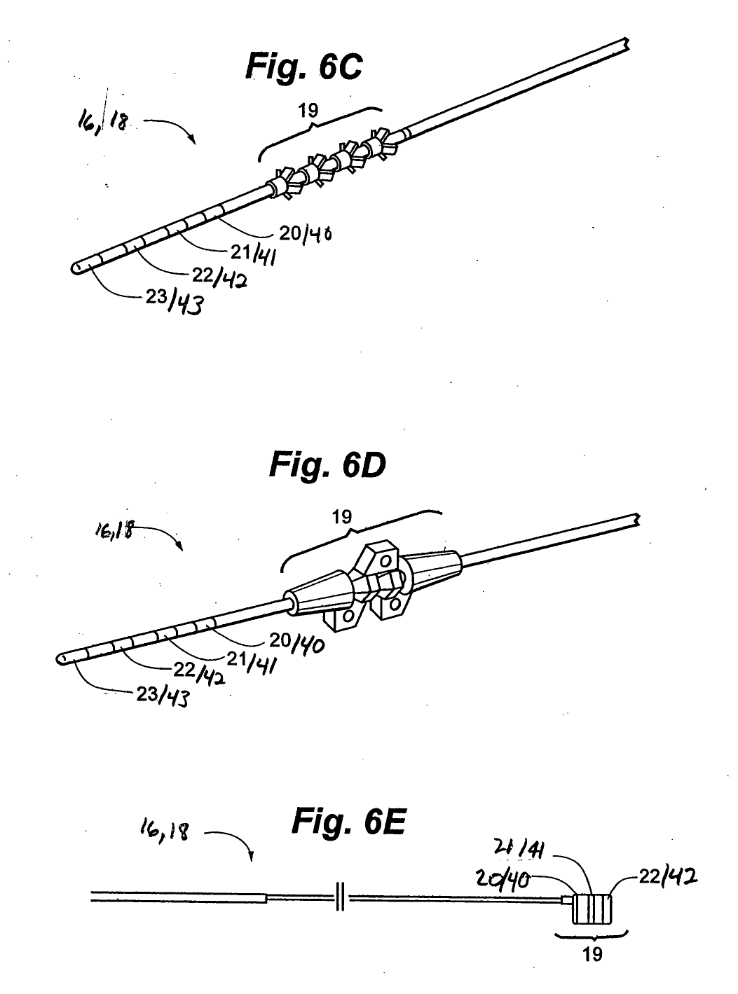

FIGS. 6A through 6E show various embodiments of a distal end of medical electrical lead 16 and/or 18 of the present invention. In FIG. 6A, lead 16/18 is a paddle lead having electrodes 20-23 arranged along an outwardly facing planar surface. Such a paddle lead 16/18 is preferably employed to stimulate peripheral nerves. In FIG. 6B, lead 16/18 is a conventional quadrapolar lead having no pre-attached anchoring mechanism 19. Electrodes 20-23 are cylindrical in shape and extend around the circumference of the lead body.

In FIG. 6C, lead 16/18 is a quadrapolar lead having tined lead anchors 19. Tines 19 may be formed from flexible or rigid biocompatible materials in accordance with the desired application. Representative examples of some tined and other types of leads suitable, adaptable or modifiable for use in conjunction with the systems, methods and devices of the present invention include those disclosed in U.S. patent application Ser. No. 10/004,732 entitled “Implantable Medical Electrical Stimulation Lead Fixation Method and Apparatus” and Ser. No. 09/713,598 entitled “Minimally Invasive Apparatus for Implanting a Sacral Stimulation Lead ” to Mamo et al., as well as those disclosed in U.S. Pat. No. 3,902,501 to Citron entitled “Endocardial Lead,” U.S. Pat. No. 4,106,512 to Bisping entitled “Transvenously Implantable Lead,” U.S. Pat. No. 5,300,107 to Stokes entitled “Universal Tined Myocardial Pacing Lead.”

In FIG. 6D, lead 16/18 is a quadrapolar lead having pre-attached suture anchor 19. In FIG. 6E, lead 16/18 is a tri-polar cuff electrode, where cuff/anchor 19 is wrapped around desired nerve or nerve portion 8 to thereby secure the distal end of lead 16/18 to the nerve and position electrodes 20-22 against or near nerve or nerve portion 25 or 26. The Medtronic Model No. 3995 cuff electrode lead is one example of a lead that may be adapted for use in the present invention, the Instructions for Use manual of which is hereby incorporated by reference herein in its entirety.

Leads 16 and 18 are preferably less than about 5 mm in diameter, and most preferably less than about 1.5 mm in diameter. Polyurethane is a preferred material for forming the lead body of leads 16 and 18, although other materials such as silicone may be employed. Electrical conductors extending between the proximal and distal ends of leads 16 and 18 for supplying electrical current to the electrodes are preferably formed of coiled, braided or stranded wires comprising an MP35N platinum-iridium alloy. Electrodes 20, 21, 22 and 23 and 40, 41, 42 and 43 may be ring electrodes, coiled electrodes, electrodes formed from portions of wire, barbs, hooks, spherically-shaped members, helically-shaped members, or may assume any of a number of different structural configurations well known in the art.

Inter-electrode distances on leads 16 and 18 are preferably about 3 mm, but other inter-electrode distances may be employed such as about 1 mm, about 2 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, about 30 mm.

Preferred surface areas of electrodes 20, 21, 22 and 23 and 40, 41, 42 and 43 range between about 1.0 sq. mm and about 100 sq. mm, between about 2.0 sq. mm and about 50 sq. mm, and about 4.0 sq. mm and about 25 sq. mm.

Preferred lengths of electrodes 20, 21, 22 and 23 and 40, 41, 42 and 43 range between about 0.25 mm and about 10 mm, between about 0.50 mm and about 8 mm, and about 1 mm and about 6 mm.

Electrodes 20, 21, 22 and 23 and 40, 41, 42 and 43 are preferably formed of platinum, although other metals and metal alloys may be employed such as stainless steel or gold.

The distal portion of lead 16 extends to a target site or position near a desired nerve or nerve portion 25 and/or 26, and is preferably held in such position by lead anchor 19. Note that lead anchor 19 may assume any of a number of different structural configurations such one or more suture sleeves, cuffs, tines, barbs, hooks, helical screws, tissue in-growth mechanisms, adhesive, polycyanoacrylate, or glue.

One, two, three, four or more electrodes 20, 21, 22 and 23 or 40, 41, 42 and 43 may be disposed at the distal end of lead 16 and/or lead 18. Electrodes 20, 21, 22 and 23 and 40, 41, 42 and 43 are preferably arranged in an axial array, although other types of arrays may be employed such as inter-lead arrays of electrodes between the distal ends of leads 16 and 18 such that nerves or nerve portions 8 disposed between leads 16 and 18 may be stimulated.

Leads 16 and 18 preferably range between about 4 inches and about 20 inches in length, and more particularly may be about 6 inches, about 8 inches, about 10 inches, about 12 inches, about 14 inches, about 16 inches or about 18 inches in length, depending on the location of the site to be stimulated and the distance of INS 10 from such site. Other lead lengths such as less than about 4 inches and more than about 20 inches are also contemplated in the present invention.

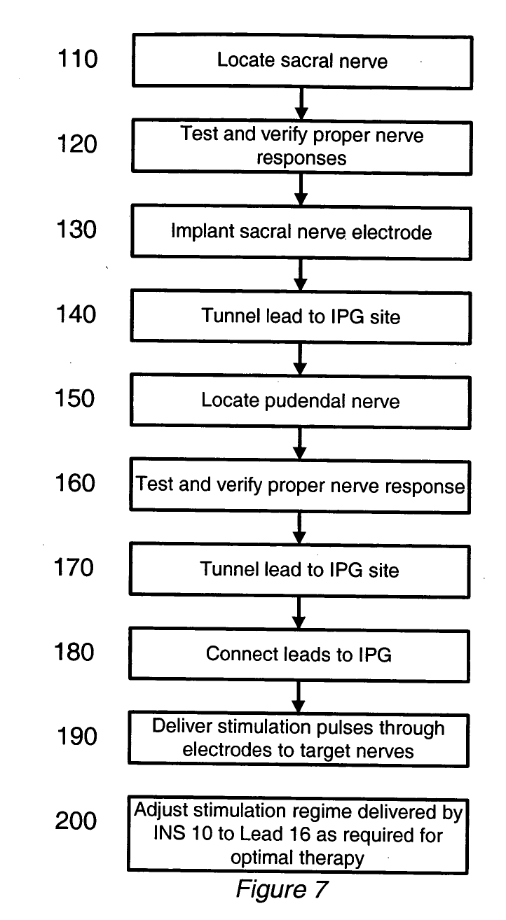

FIG. 7 shows a flow diagram according to one embodiment of a method of the present invention for stimulating sacral and pudendal nerves or nerve portions 25 and 26 . Note that the order in which the steps shown in FIG. 7 are carried out may be changed, and that the resulting method will nevertheless fall within the scope of the present invention. In FIG. 7, at step 110 one or more desired sacral nerve stimulation locations is located. Various techniques such as visualization under fluoroscopy or the use of anatomical landmarks may be used to locate the sacral nerve or nerve portion 25 to be stimulated. Step 120 is employed to test and verify proper sacral nerve or nerve portion 25 response prior to implantation of the stimulation electrode. An electrical signal delivered to the nerve through a needle is typically employed to elicit such a nerve response. The nerve response may be detected through a motor response that may be visually detected, a sensory response as reported by the patient or through an electrical response. Following verification of proper nerve response, at step 130 the sacral nerve electrode is implanted. In step 140, INS 10 is implanted in an appropriate location within the patient such that the proximal end of lead 16 or 18 may be operably connected thereto, and such that INS 10 is placed in such a location that discomfort and the risk of infection to the patient are minimized.

Step 150 in FIG. 7 is employed to locate the pudendal nerve. Location of pudendal nerve or nerve portion 26 is typically done through the identification of anatomical landmarks. Palpation of the patient as well as usage of fluoroscopy, x-ray and EMG may be employed to assist in proper location of the pudendal nerve. Step 170 is employed to implant INS 10 in an appropriate location within the patient such that the proximal end of lead 16 may be operably connected thereto and such that INS 10 is placed in such a location that discomfort and the risk of infection to the patient are minimized. Step 180 is used to operably connect INS 10 to lead 16 or 18, which may or may not require the use of optional lead extension 15 and lead connector 13. In Step 190, INS 10 is activated and stimulation pulses are delivered to electrodes 20, 21, . . . n or 40, 41, . . . n through lead 16 or 18 to the desired nerve stimulation location 25 or 26. In step 200, the electrical pulse stimulation parameters are adjusted to optimize the therapy delivered to the patient. Such adjustment may entail one or more of adjusting the number or configuration of electrodes or leads used to stimulate the selected location, pulse amplitude, pulse frequency, pulse width, pulse morphology (e.g., square wave, triangle wave, sinusoid, biphasic pulse, tri-phasic pulse, etc.), times of day or night when pulses are delivered, pulse cycling times, the positioning of the lead or leads, and/or the enablement or disablement of “soft start” or ramp functions respecting the stimulation regime to be provided. Note that methods of the present invention further contemplate the placement and implantation of multiple leads.

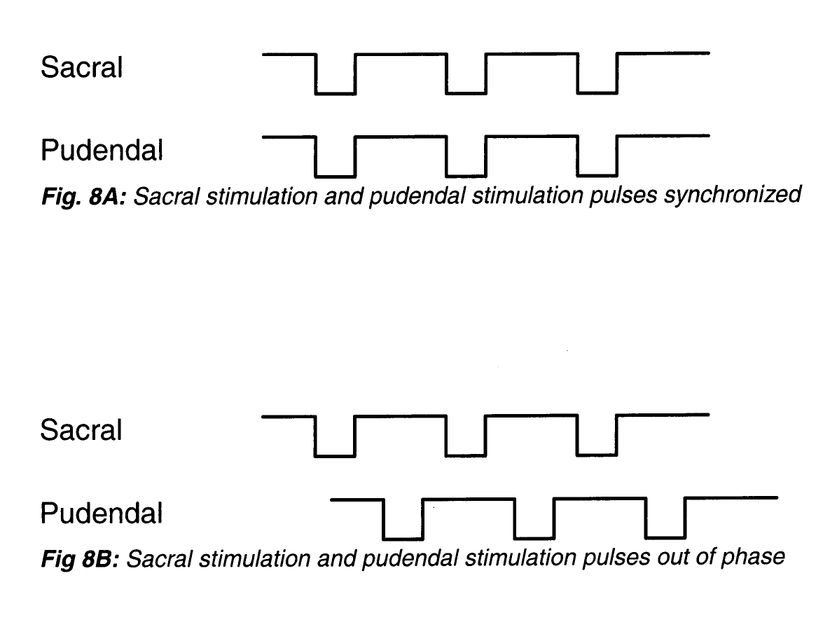

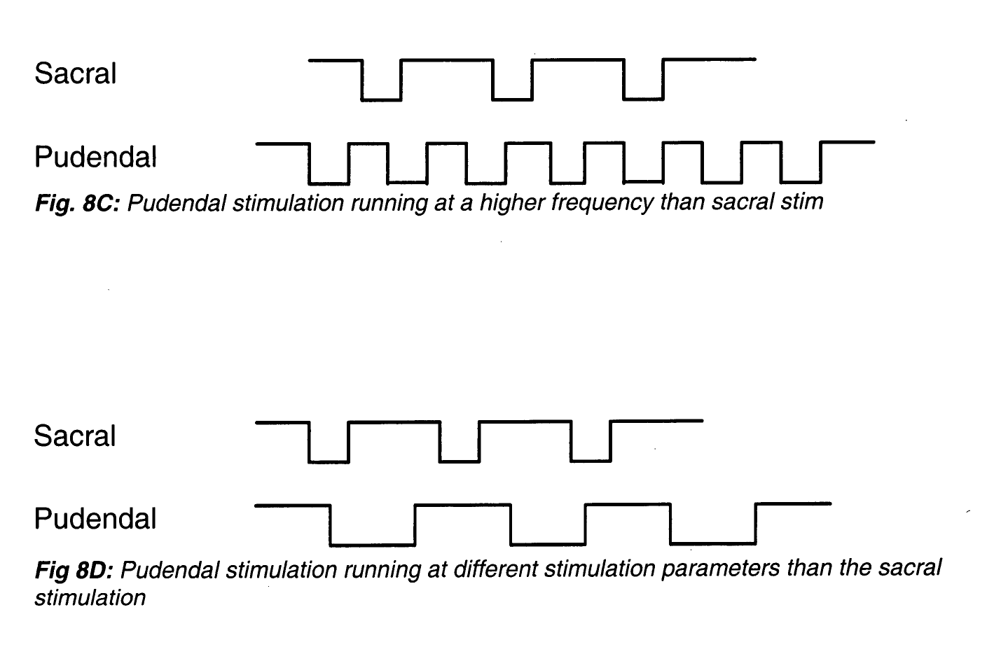

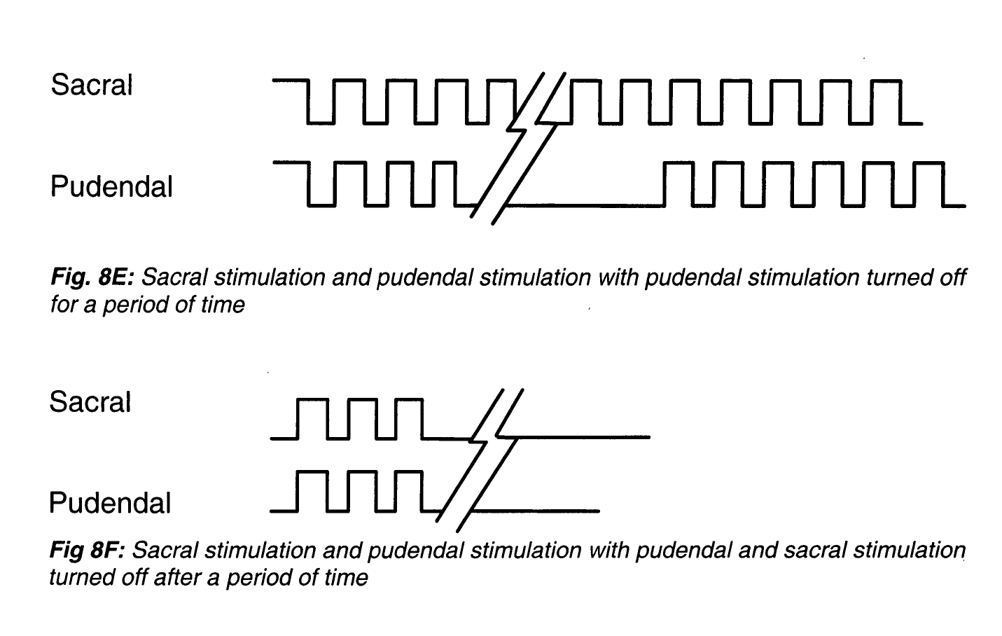

FIGS. 8A through 8F shows various embodiments of the first and second pulse regimes of the present invention. In FIG. 8A, electrical pulses delivered to sacral nerve or nerve portion 25 are synchronized with the electrical pulses delivered to pudendal nerve or nerve portion 26. In FIG. 8B, electrical pulses delivered to sacral nerve or nerve portion 25 are out of phase in respect of the electrical pulses delivered to pudendal nerve or nerve portion 26. In FIG. 8B, the electrical pulses delivered to sacral nerve or nerve portion 25 have a lower frequency than the electrical pulses delivered to pudendal nerve or nerve portion 26. In FIG. 8D, electrical pulses delivered to sacral nerve or nerve portion 25 are characterized in having different stimulation parameters than those of the electrical pulses delivered to pudendal nerve or nerve portion 26. In FIG. 8E, electrical pulses are delivered without interruption and on a continuous basis to sacral nerve or nerve portion 25, while the delivery of electrical pulses to pudendal nerve or nerve portion 26 is suspended for a period of time. In FIG. 8F, electrical pulses are delivered to sacral nerve or nerve portion 25 and pudendal nerve or nerve portion 26, but at a certain point in time are suspended for a predetermined period of time or until a sensed quantity changes in a predetermined fashion.

Some examples of dual stimulation techniques falling within the scope of the present invention are as follows:
One pulse regime may be delivered continuously, while the other pulse regime is turned on and off in accordance with the patient's symptoms, as such symptoms may wax and wane;
Both pulse regimes may be delivered continuously, one intermittently and the other continuously, or both intermittently;
The two pulse regimes may be different in respect of amplitude, pulse-width, frequency, pulse morphology, and the like;
One or both pulse regimes may be delivered according to a scheduled or detected activity, or according to a predetermined or detected schedule, the activity or schedule having one or more pulse regimes associated therewith, the activities or schedules comprising one or more of circadian rhythms, daytime or nighttime activities or schedules, meals, periods of exercise, periods of sleep, sexual activity, and the like;
One pulse regime may be delivered in accordance with a preprogrammed regime, while the other pulse regime may be delivered, activated, modified and/or terminated via patient activation, modification or termination;
One or both pulse regimes may be delivered between different leads having one or more electrodes each to obtain a spatially broad stimulation pattern;
One or both pulse regimes may be controlled, activated and/or terminated by the patient to customize the delivered therapy.

Some representative ranges of preferred electrical pulse stimulation parameters capable of being delivered by INS 10 through leads 16 and/or 18 include the following:

Frequency: Between about 50 Hz and about 100 Hz;
Between about 10 Hz and about 250 Hz; and
Between about 0.5 Hz and about 500 Hz.

Amplitude: Between about 1 Volt and about 10 Volts;
Between about 0.5 Volts and about 20 Volts; and
Between about 0.1 Volts and about 50 Volts.

Pulse Width: Between about 180 microseconds and about 450 microseconds;
Between about 100 microseconds and about 1000 microseconds; and
Between about 10 microseconds and about 5000 microseconds.

In the event multiple signals are employed to stimulate a desired site, the spatial and temporal phase between the signals may be adjusted or varied to produce the desired stimulation pattern or sequence. That is, in the present invention beam forming and specific site targeting via electrode array adjustments are specifically contemplated. Electrode configurations, arrays and stimulation patterns and methods similar to those disclosed by Holsheimer in U.S. Pat. No. 6,421,566 entitled “Selective Dorsal Column Stimulation in SCS, Using Conditioning Pulses,” U.S. Pat. No. 5,643,330 entitled “Multichannel Apparatus for Epidural Spinal Cord Stimulation” and U.S. Pat. No. 5,501,703 entitled “Multichannel Apparatus for Epidural Spinal Cord Stimulator,” the respective entireties of which are hereby incorporated by reference herein, may also be adapted or modified for use in the present invention. Electrode configurations, arrays, leads, stimulation patterns and methods similar to those disclosed by Thompson in U.S. Pat. No. 5,800,465 entitled “System and Method for Multi-site Steering of Cardiac Stimuli,” the entirety of which is hereby incorporated by reference herein, may also be adapted or modified for use in the present invention to permit the steering of electrical fields. Thus, although FIG. 1 shows four electrodes located at the distal end of lead 16 near sacral nerve S3, other lead locations and electrode configurations are possible and contemplated in the present invention.

In addition, in the present invention it is contemplated that drugs be delivered to specific sites within a patient using well known fully implantable drug pump devices in combination with providing electrical stimulation to the nerves or nerve portions described above. According to such a method, the drug pump may be incorporated into the same housing as INS 10, or be separate therefrom in its own hermetically sealed housing. The drug catheter attached to the implantable drug pump through which the drug is delivered to the specific site may also be incorporated into lead 16 or 18, or may be separate therefrom. Drugs or therapeutic agents delivered in accordance with this method include, but are not limited to, antibiotics, pain relief agents such as demerol and morphine, radioactive or radio-therapeutic substances or agents for killing or neutralizing maycer cells, genetic growth factors for encouraging the growth of healthy tissues, drugs for facilitating or encouraging penile or clitoral engorgement, and the like.

Also hereby incorporated by reference herein in its entirety is U.S. patent application Ser. No. 20020082665A1 to Haller et al. published Jun. 27, 2002 entitled “System and Method of Communicating between an Implantable Medical Device and a Remote Computer System or Health Care Provider.” In the present invention it is further contemplated that the methods and devices described hereinabove be extended to include the various communication systems of Haller et al. for at least one of monitoring the performance of INS 10 and/or an implantable drug pump implanted within the body of a patient, monitoring the health of the patient and remotely delivering an electrical stimulation and/or drug therapy to the patient through INS 10 and/or the optional implantable drug pump, INS 10 or the implantable drug pump being capable of bi-directional communication with a communication module located external to the patient's body, the system comprising: (a) INS 10 and optionally the implantable drug pump; (b) the communication module; (c) a mobile telephone or similar device operably connected to the communication module and capable of receiving information therefrom or relaying information thereto; (e) a remote computer system, and (f) a communication system capable of bidirectional communication.

The preceding specific embodiments are illustrative of the practice of the invention. It is to be understood, therefore, that other expedients known to those skilled in the art or disclosed herein may be employed without departing from the invention or the scope of the appended claims.

In the claims, means plus function clauses are intended to cover the structures described herein as performing the recited function and their equivalents. Means plus function clauses in the claims are not intended to be limited to structural equivalents only, but are also intended to include structures which function equivalently in the environment of the claimed combination. All printed publications and patents referenced hereinabove are hereby incorporated by referenced herein, each in its respective entirety.

1. A method of treating at least one diagnosed pelvic floor disorder in a patient, the at least one disorder being selected from the group consisting of urinary voiding dysfunction, fecal voiding dysfunction, constipation, stress incontinence, urge incontinence, urinary retention disorder, sexual dysfunction, orgasmic dysfunction, erectile dysfunction, pelvic pain, prostatitis, prostatalgia and prostatodynia, the method comprising:
providing an hermetically sealed implantable electrical pulse generator configured to provide at least first and second electrical stimulation pulse regimes via at least first and second implantable medical electrical leads;
providing the first implantable medical electrical lead, the first lead being configured for implantation adjacent, around or in at least one of a sacral nerve or branches or portions thereof, the first lead comprising proximal and distal ends and at least a first electrode;
providing the second implantable medical electrical lead, the second lead being configured for implantation adjacent a pudendal nerve or branches or portions thereof, the second lead comprising proximal and distal ends and at least a second electrode;
implanting the first lead in or near a first tissue volume of the patient adjacent, around or in one of the sacral nerve or branches or portions thereof;
implanting the second lead in or near a second tissue volume of the patient adjacent, around or in one of the pudendal nerve or branches or portions thereof; operably connecting the proximal end of the first lead to the implantable pulse generator;
operably connecting the proximal end of the second lead to the implantable pulse generator;
implanting the implantable pulse generator within the patient; and delivering, from the implantable pulse generator, first electrical stimulation pulses to or near at least portions of the first tissue volume through the first lead and at least the first electrode, the first pulses being provided in accordance with the first electrical stimulation pulse regime;
delivering, from the implantable pulse generator, second electrical stimulation pulses to or near at least portions of the second tissue volume through the second lead and at least the second electrode, the second pulses being provided in accordance with the second electrical stimulation pulse regime;
wherein the combination of the first and the second electrical pulse regimes delivered through the first and second leads to or near at least portions of the first and second tissue volumes provides to the patient at least partial relief from the pelvic floor disorder.

2. The method of claim 1, wherein at least one of the first lead and the second lead is selected from the group consisting of a unipolar lead, a bipolar lead, a tri-polar lead, a quadrapolar lead, and a multi-polar lead.

3. The method of claim 1, wherein at least one of the first lead and the second lead is selected from the group consisting of a beam steering lead comprising multiple electrodes and a lead comprising multiple electrodes disposed in an areal pattern on a planar or curved surface.

4. The method of claim 1, wherein at least one of the first lead and the second lead is selected from the group consisting of a cuff lead, a paddle lead, a tined lead, a lead having an active fixation device or member disposed thereon, attached thereto or forming a portion thereof.

5. The method of claim 1, wherein at least one of the first lead and the second lead comprises a fixation mechanism selected from the group consisting of a suture sleeve, a barb, a helical screw, a hook and a tissue in-growth mechanism.

6. The method of claim 1, wherein at least one of the first lead and the second lead further comprises one or more electrodes configured to operate in conjunction with an electrically conductive portion of the implantable pulse generator acting as an indifferent electrode.

7. The method of claim 1, further comprising delivering electrical pulses through a third tissue volume disposed near or between the electrodes located on the first and second leads.

8. The method of claim 1, wherein the electrical stimulation pulses that are delivered to the first and second tissue volumes cause paresthesia, or the masking or blocking pain signals originating in or carried by a desired or target nerve or nerve portion.

9. The method of claim 1, further comprising providing a lead extension, operably connecting same between one of the proximal end of the at least first lead and the proximal end of the at least first lead, and the implantable pulse generator, and delivering the electrical stimulation pulses through the lead extension.

10. The method of claim 1, wherein at least one of the first lead and the second lead is selected from the group consisting of a lead comprising a lead body less than about 5 mm in diameter, a lead comprising a lead body less than about 1.5 mm in diameter, a lead having a lead body comprising polyurethane or silicone, a lead comprising electrical conductors disposed within the body thereof and extending between the proximal and distal ends of the lead wherein the conductors are formed of coiled, braided or stranded wires, and a lead comprising at least one ring electrode, at least one coiled electrode, at least one button electrode, at least one electrode formed from a portion of wire, a cuff, a barb or a hook, a spherically-shaped electrode, and a helically-shaped electrode.

11. The method of claim 1, wherein an inter-electrode distance of at least one of the first lead and the second lead is selected from the group consisting of about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, and about 30 mm.

12. The method of claim 1, wherein the at least one electrode of at least one of the first lead and the second lead has an electrode surface area ranging between about 1.0 sq. mm and about 100 sq. mm, between about 2.0 sq. mm and about 50 sq. mm, or between about 4.0 sq. mm and about 25 sq. mm.

13. The method of claim 1, wherein the distance between the proximal and distal ends of at least one of the first lead and the second lead is selected from the group consisting of less than about 4 inches, about 4 inches, about 6 inches, about 8 inches, about 10 inches, about 12 inches, about 14 inches, about 16 inches about 18 inches, about 20 inches and more than about 20 inches.

14. The method of claim 1, wherein the implantable pulse generator comprises an electronic circuitry architecture selected from the group consisting of a microprocessor-based architecture, a logic architecture and a state machine architecture.

15. The method of claim 1, further comprising providing an external programming unit and effecting telemetric communication between the programming unit and the implantable pulse generator.

16. The method of claim 1, wherein the implantable pulse generator further comprises at least one of a primary battery power source and a secondary battery power source.

17. The method of claim 1, wherein the implantable pulse generator is configurable so as to permit at least one of the frequency, rate, amplitude, phase, width and morphology of the pulses generated and delivered by the implantable pulse generator to be varied programmably by a user.

18. The method of claim 1, wherein at least one of the first lead and the second lead is configured for percutaneous introduction and implantation within the patient.

19. The method of claim 1, wherein the implantable pulse generator and at least one of the first lead and the second lead are capable of generating and delivering electrical pulses having frequencies ranging between about 50 Hz and about 100 Hz, between about 10 Hz and about 250 Hz, and between about 0.5 Hz and about 500 Hz.

20. The method of claim 1, wherein the implantable pulse generator and at least one of the first lead and the second lead are capable of generating and delivering electrical pulses having amplitudes ranging between about 1 Volt and about 10 Volts, between about 0.5 Volts and about 20 Volts, and between about 0.1 Volts and about 50 Volts.

21. The method of claim 1, wherein the implantable pulse generator and at least one of the first lead and the second lead are capable of generating and delivering electrical pulses having pulse widths ranging between about 180 microseconds and about 450 microseconds, between about 100 microseconds and about 1000 microseconds, and between about 10 microseconds and about 5000 microseconds.

22. The method of claim 1, wherein the implantable pulse generator and at least one of the first lead and the second lead are capable of generating and delivering electrical pulses having varying spatial or temporal phases.

23. The method of claim 1, further comprising delivering a drug to the patient.

24. The method of claim 23, further comprising providing, implanting and activating an implantable drug pump for providing the drug to the patient.

25. The method of claim 1, wherein at least one of activation, modification and termination of at least one of the first pulse regime and the second pulse regime is carried out by the patient or a health care giver.

26. The method of claim 25, wherein the at least one of activation, modification and termination of at least one of the first pulse regime and the second pulse regime is carried out in response to patient symptoms appearing or disappearing, or the patient feeling or not feeling symptoms

27. The method of claim 25, wherein patient or health care giver activation, modification and/or termination of the first or second pulse regime is accomplished through infra-red, telemetric, radio, magnetic, or ultrasonic means.

28. The method of claim 1, wherein the first pulse regime is delivered while delivery of the second pulse regime is initiated later in response to a sensed physical parameter or symptom.

29. The method of claim 1, wherein the first and second pulse regimes are initially delivered, and delivery of at least one of the first and second pulse regimes is subsequently terminated or modified.

30. The method of claim 1, wherein at least one of the first pulse regime and the second pulse regime is one of activated, modified and terminated in response to a physical parameter or symptom being sensed.

31. The method of claim 30, wherein the physical parameter is sensed using a sensor selected from the group consisting of a bladder pressure sensor, a leak sensor, a volume sensor, a urinary volume or pressure sensor, a urinary impedance sensor, a nerve electrical signal sensor, and an electromyographic sensor.

32. A method of treating at least one diagnosed pelvic floor disorder in a patient, the at least one disorder being selected from the group consisting of urinary voiding dysfunction, fecal voiding dysfunction, constipation, stress incontinence, urge incontinence, urinary retention disorder, sexual dysfunction, orgasmic dysfunction, erectile dysfunction, pelvic pain, prostatitis, prostatalgia and prostatodynia, the method comprising:
providing a first hermetically sealed implantable electrical pulse generator configured to provide at least a first electrical stimulation pulse regime via at least a first implantable medical electrical lead;
providing a second hermetically sealed implantable electrical pulse generator configured to provide at least a second electrical stimulation pulse regime via at least a second implantable medical electrical lead;
providing the first implantable medical electrical lead, the first lead being configured for implantation adjacent, around or in at least one of a sacral nerve or branches or portions thereof, the first lead comprising proximal and distal ends and at least a first electrode;
providing the second implantable medical electrical lead, the second lead being configured for implantation adjacent a pudendal nerve or branches or portions thereof, the second lead comprising proximal and distal ends and at least a second electrode;
implanting the first lead in or near a first tissue volume of the patient adjacent, around or in one of the sacral nerve or branches or portions thereof;
implanting the second lead in or near a second tissue volume of the patient adjacent, around or in one of the pudendal nerve or branches or portions thereof;
operably connecting the proximal end of the first lead to the first implantable pulse generator;
operably connecting the proximal end of the second lead to the second implantable pulse generator;
implanting the first implantable pulse generator within the patient;
implanting the second implantable pulse generator within the patient; and
delivering, from the first implantable pulse generator, first electrical stimulation pulses to or near at least portions of the first tissue volume through the first lead and at least the first electrode, the first pulses being provided in accordance with the first electrical stimulation pulse regime;
delivering, from the second implantable pulse generator, second electrical stimulation pulses to or near at least portions of the second tissue volume through the second lead and at least the second electrode, the second pulses being provided in accordance with the second electrical stimulation pulse regime;
wherein the combination of the first and the second electrical pulse regimes delivered through the first and second leads to or near at least portions of the first and second tissue volumes provides to the patient at least partial relief from the pelvic floor disorder.

33. The method of claim 32, wherein at least one of the first lead and the second lead is selected from the group consisting of a unipolar lead, a bipolar lead, a tri-polar lead, a quadrapolar lead, and a multi-polar lead.

34. The method of claim 32, wherein at least one of the first lead and the second lead is selected from the group consisting of a beam steering lead comprising multiple electrodes and a lead comprising multiple electrodes disposed in an areal pattern on a planar or curved surface.

35. The method of claim 32, wherein at least one of the first lead and the second lead is selected from the group consisting of a cuff lead, a paddle lead, a tined lead, a lead having an active fixation device or member disposed thereon, attached thereto or forming a portion thereof.

36. The method of claim 32, wherein at least one of the first lead and the second lead comprises a fixation mechanism selected from the group consisting of a suture sleeve, a cuff, a barb, a helical screw, a hook and a tissue in-growth mechanism.

37. The method of claim 32, wherein at least one of the first lead and the second lead further comprises one or more electrodes configured to operate in conjunction with an electrically conductive portion of the implantable pulse generator acting as an indifferent electrode.

38. The method of claim 32, further comprising delivering electrical pulses through a third tissue volume disposed near or between the electrodes located on the first and second leads.

39. The method of claim 32, wherein the electrical stimulation pulses that are delivered to the first and second tissue volumes cause paresthesia, or the masking or blocking pain signals originating in or carried by a desired or target nerve or nerve portion.

40. The method of claim 32, further comprising providing a lead extension, operably connecting same between one of the proximal end of the at least first lead and the proximal end of the at least first lead, and the implantable pulse generator, and delivering the electrical stimulation pulses through the lead extension.

41. The method of claim 32, wherein at least one of the first lead and the second lead is selected from the group consisting of a lead comprising a lead body less than about 5 mm in diameter, a lead comprising a lead body less than about 1.5 mm in diameter, a lead having a lead body comprising polyurethane or silicone, a lead comprising electrical conductors disposed within the body thereof and extending between the proximal and distal ends of the lead wherein the conductors are formed of coiled, braided or stranded wires, and a lead comprising at least one ring electrode, at least one coiled electrode, at least one button electrode, at least one electrode formed from a portion of wire, a barb or a hook, a spherically-shaped electrode, and a helically-shaped electrode.

42. The method of claim 32, wherein an inter-electrode distance of at least one of the first lead and the second lead is selected from the group consisting of about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, and about 30 mm.

43. The method of claim 32, wherein the at least one electrode of at least one of the first lead and the second lead has an electrode surface area ranging between about 1.0 sq. mm and about 100 sq. mm, between about 2.0 sq. mm and about 50 sq. mm, or between about 4.0 sq. mm and about 25 sq. mm.

44. The method of claim 32, wherein the distance between the proximal and distal ends of at least one of the first lead and the second lead is selected from the group consisting of less than about 4 inches, about 4 inches, about 6 inches, about 8 inches, about 10 inches, about 12 inches, about 14 inches, about 16 inches about 18 inches, about 20 inches and more than about 20 inches.

45. The method of claim 32, wherein the implantable pulse generator comprises an electronic circuitry architecture selected from the group consisting of a microprocessor-based architecture, a logic architecture and a state machine architecture.

46. The method of claim 32, further comprising providing an external programming unit and effecting telemetric communication between the programming unit and the implantable pulse generator.

47. The method of claim 32, wherein the implantable pulse generator further comprises at least one of a primary battery power source and a secondary battery power source.

48. The method of claim 32, wherein the implantable pulse generator is configurable so as to permit at least one of the frequency, rate, amplitude, phase, width and morphology of the pulses generated and delivered by the implantable pulse generator to be varied programmably by a user.

49. The method of claim 32, wherein at least one of the first lead and the second lead is configured for percutaneous introduction and implantation within the patient.

50. The method of claim 32, wherein the implantable pulse generator and at least one of the first lead and the second lead are capable of generating and delivering electrical pulses having frequencies ranging between about 50 Hz and about 100 Hz, between about 10 Hz and about 250 Hz, and between about 0.5 Hz and about 500 Hz.

51. The method of claim 32, wherein the implantable pulse generator and at least one of the first lead and the second lead are capable of generating and delivering electrical pulses having amplitudes ranging between about 1 Volt and about 10 Volts, between about 0.5 Volts and about 20 Volts, and between about 0.1 Volts and about 50 Volts.

52. The method of claim 32, wherein the implantable pulse generator and at least one of the first lead and the second lead are capable of generating and delivering electrical pulses having pulse widths ranging between about 180 microseconds and about 450 microseconds, between about 100 microseconds and about 1000 microseconds, and between about 10 microseconds and about 5000 microseconds.

53. The method of claim 32, wherein the implantable pulse generator and at least one of the first lead and the second lead are capable of generating and delivering electrical pulses having varying spatial or temporal phases.

54. The method of claim 32, further comprising delivering a drug to the patient.

55. The method of claim 54, further comprising providing, implanting and activating an implantable drug pump for providing the drug to the patient.
